(19) United States
(12) Plant Patent Application Publication
(10) Pub. No.: US 2011/0055990 P1
(43) Pub. Date: Mar. 3, 2011

(54) Mushroom plants named 'PLE-3' and 'PLE-5'

(50) ;

(75) Inventors: Eriko Kurashima, Nagano-shi (JP); Satoshi Inatomi, Nagano-shi (JP)

(73) Assignee: Hokuto Corporation

(21) Appl. No.: 12/585,095

(22) Filed: Sep. 3, 2009

(51) Int. Cl. A01H 15/00; C12N 1/14

(52) U.S. Cl. PLT/394; 435/254.1

(57) ABSTRACT

The present invention relates to new, distinct horticultural varieties of mushroom plant of the species Pleurotus eryngii. The new varieties, named ‘PLE-3’ and ‘PLE-5’, was found by crossbreeding eryngii varieties having dominant traits. The varieties are featured by its presentable carpophore, delicacy, stability and uniformity and pronounced distinguishability.

### VARIETAL DENOMINATION

Latin name of the genus and species of the plant claimed: Pleurotus Eryngii

Variety denomination: Eryngii ‘PLE-3’ and Eryngii ‘PLE-5’

### BACKGROUND OF THE INVENTION

The present invention relates to a new and distinct cultivar of mushroom plant of the species Pleurotus eryngii. Particularly, this invention relates to a new cultivar of eryngii, which is produced by crossbreeding eryngii varieties having dominant traits and featured by its presentable carpophore, delicacy, stability and uniformity and pronounced distinguishability.

Bottle cultivation has started around 1993 in Japan to produce the eryngii. In 2008, 38000 tons of eryngii was produced in Japan. The eryngii grows wild in North America, but it has been sparsely cultivated by a cultivation method using a bag and is in slight demand as food. Recently, bottle cultivation for producing the eryngii on a factory scale has started. Since it has been learned that the eryngii is low in calories and has beneficial effects of preventing fatty liver, future increase in demand for eryngii is expected.

However, the modern cultivar of eryngii is good in quality, but it has little adaptability to cultivation conditions such as a culture medium type in the U.S. As an example, a cultivating cycle are somewhat lengthened, thus posing a problem to industrial yieldability.

### SUMMARY OF THE INVENTION

In view of the foregoing disadvantages inherent in the known types of eryngii now present in the prior art, for the purpose of solving problems in cultivating the modern cultivar of eryngii and preserving the quality as well or better than the modern cultivar, the present invention provides mushroom plants named ‘PLE-3’ and ‘PLE-5’ cultivated and produced by crossbreeding parent strains ‘PLE-2’ and ‘MH006376’ having superior properties of stability, uniformity and distinguishability.

The successfully cultivated mushrooms named ‘PLE-3’ and ‘PLE-5’ of the invention are the same strain created by crossbreeding the same parents, so to speak, brothers. These two strains have the closely similar characteristics.

### BRIEF DESCRIPTION OF THE FIGURES

FIGS. 1(A) and 1(B) are photographs showing the top and bottom of dual culture of two ‘PLE-3’ strains of the invention.

FIGS. 2(A) and 2(B) are photographs showing the top and bottom of dual culture of two ‘PLE-5’ strains of the invention.

FIGS. 3(A) and 3(B) are photographs showing the top and bottom of dual culture of ‘PLE-3’ and ‘PLE-5’ of strains the invention.

FIGS. 4(A) and 4(B) are photographs showing the top and bottom of dual culture of ‘PLE-3’ strain of the invention and parent strain ‘PLE-2’.

FIGS. 5(A) and 5(B) are photographs showing the top and bottom of dual culture of ‘PLE-3’ strain of the invention and parent strain ‘PLE-2’.

FIGS. 6(A) and 6(B) are photographs showing the top and bottom of dual culture of ‘PLE-3’ strain of the invention and strain ‘ATCC32047’.

FIGS. 7(A) and 7(B) are photographs showing the top and bottom of dual culture of ‘PLE-3’ strain of the invention and strain ‘ATCC32047’.

FIGS. 8(A) and 8(B) are photographs showing the top and bottom of dual culture of ‘PLE-3’ strain of the invention and strain ‘ATCC90212’.

FIGS. 9(A) and 9(B) are photographs showing the top and bottom of dual culture of ‘PLE-3’ strain of the invention and strain ‘ATCC90212’.

FIGS. 10(A) and 10(B) are photographs showing the top and bottom of dual culture of ‘PLE-3’ strain of the invention and strain ‘ATCC90787’.

FIGS. 11(A) and 11(B) are photographs showing the top and bottom of dual culture of ‘PLE-5’ strain of the invention and strain ‘ATCC90787’.

FIGS. 12(A) and 12(B) are photographs showing the top and bottom of dual culture of ‘PLE-3’ strain of the invention and strain ‘ATCC90887’.

FIGS. 13(A) and 13(B) are photographs showing the top and bottom of dual culture of ‘PLE-5’ strain of the invention and strain ‘ATCC90887’.

FIGS. 14(A) and 14(B) are photographs showing the top and bottom of dual culture of ‘PLE-3’ strain of the invention and strain ‘ATCC90888’.

FIGS. 15(A) and 15(B) are photographs showing the top and bottom of dual culture of ‘PLE-5’ strain of the invention and strain ‘ATCC90888’.

FIGS. 16(A) and 16(B) are photographs showing the top and bottom of dual culture of ‘PLE-3’ strain of the invention and strain ‘ATCC96054’.

FIGS. 17(A) and 17(B) are photographs showing the top and bottom of dual culture of ‘PLE-5’ strain of the invention and strain ‘ATCC96054’.

FIGS. 18(A) and 18(B) are photographs showing the top and bottom of dual culture of ‘PLE-3’ strain of the invention and strain ‘MH006062’.

FIGS. 19(A) and 19(B) are photographs showing the top and bottom of dual culture of ‘PLE-5’ strain of the invention and strain ‘MH006062’.

FIGS. 20(A) and 20(B) are photographs showing the top and bottom of dual culture of ‘PLE-3’ strain of the invention and strain ‘MH006200’.

FIGS. 21(A) and 21(B) are photographs showing the top and bottom of dual culture of ‘PLE-5’ strain of the invention and strain ‘MH006200’.

FIGS. 22(A) and 22(B) are photographs showing the top and bottom of dual culture of ‘PLE-3’ strain of the invention and strain ‘MH006202’.

FIGS. 23(A) and 23(B) are photographs showing the top and bottom of dual culture of ‘PLE-5’ strain of the invention and strain ‘MH006202’.

FIGS. 24(A) and 24(B) are photographs showing the top and bottom of dual culture of ‘PLE-3’ strain of the invention and strain ‘MH006207’.

FIGS. 25(A) and 25(B) are photographs showing the top and bottom of dual culture of ‘PLE-5’ strain of the invention and strain ‘MH006207’.

FIGS. 26(A) and 26(B) are photographs showing the top and bottom of dual culture of ‘PLE-3’ strain of the invention and strain ‘MH006322’.

FIGS. 27(A) and 27(B) are photographs showing the top and bottom of dual culture of ‘PLE-5’ strain of the invention and strain ‘MH006322’.

FIGS. 28(A) and 28(B) are photographs showing the top and bottom of dual culture of ‘PLE-3’ strain of the invention and strain ‘MH006219’.

FIGS. 29(A) and 29(B) are photographs showing the top and bottom of dual culture of ‘PLE-5’ strain of the invention and strain ‘MH006219’.

FIGS. 30(A) and 30(B) are photographs showing the top and bottom of dual culture of ‘PLE-3’ strain of the invention and strain ‘MH006242’.

FIGS. 31(A) and 31(B) are photographs showing the top and bottom of dual culture of ‘PLE-5’ strain of the invention and strain ‘MH006242’.

FIGS. 32(A) and 32(B) are photographs showing the top and bottom of dual culture of ‘PLE-3’ strain of the invention and strain ‘MH006279’.

FIGS. 33(A) and 33(B) are photographs showing the top and bottom of dual culture of ‘PLE-5’ strain of the invention and strain ‘MH006279’.

FIG. 34 is a photograph showing carpophore of ‘PLE-3’ strain of the invention in a culturing state in a characteristic assay.

FIG. 35 is a photograph showing carpophore of ‘PLE-5’ strain of the invention in a culturing state in a characteristic assay.

FIG. 36 is a photograph showing carpophore of parent strain ‘PLE-2’ in a culturing state in a characteristic assay.

FIG. 37 is a photograph showing carpophore of ‘PLE-3’ strain of the invention under normal cultivation.

FIG. 38 is a photograph showing carpophore of ‘PLE-5’ strain of the invention under normal cultivation.

FIG. 39 is a photograph showing carpophore of parent strain ‘PLE-2’ under normal cultivation.

### DETAILED DESCRIPTION OF THE INVENTION

The present invention relates to a new and distinct cultivar of mushrooms named ‘PLE-3’ and ‘PLE-5’ belonging to Pleurotus eryngii, featured by its presentable carpophore, delicacy, stable and consistent productivity and pronounced distinguishability. The mushrooms ‘PLE-3’ and ‘PLE-5’ of the invention are cultivated in the following cultivation processes.

### PROCESSES OF CULTIVATION OF PLE-3 AND PLE-5

July 2004: Strain MH006367 having dominant trait was produced by crossbreeding parent strains HOKUTO PLE-2 and MH006220.

February 2007: Strain MH006376 was produced by crossbreeding an existing strain MH006322 and the aforementioned strain MH006367.

December 2007: Varieties PLE-3 and PLE-5 were produced by crossbreeding the aforementioned HOKUTO PLE-2 and MH006376, and then, a cultivation experiment and a characteristic assay were made about the varieties PLE-3 and PLE-5 thus obtained.

August 2008: The cultivation of PLE-3 and PLE-5 was concluded after confirmation of distinguishability, stability and uniformity of the strains.

September 2008: With respect to the new varieties PLE-3 and PLE-5, applications for plant variety registration were filed with Agriculture, Forestry and Fisheries Ministry of Japan and accepted on 5 Sep. 2008.

### CHARACTERISTICS OF PLE-3 AND PLE-5

(1) Feature comparison with existing varieties:

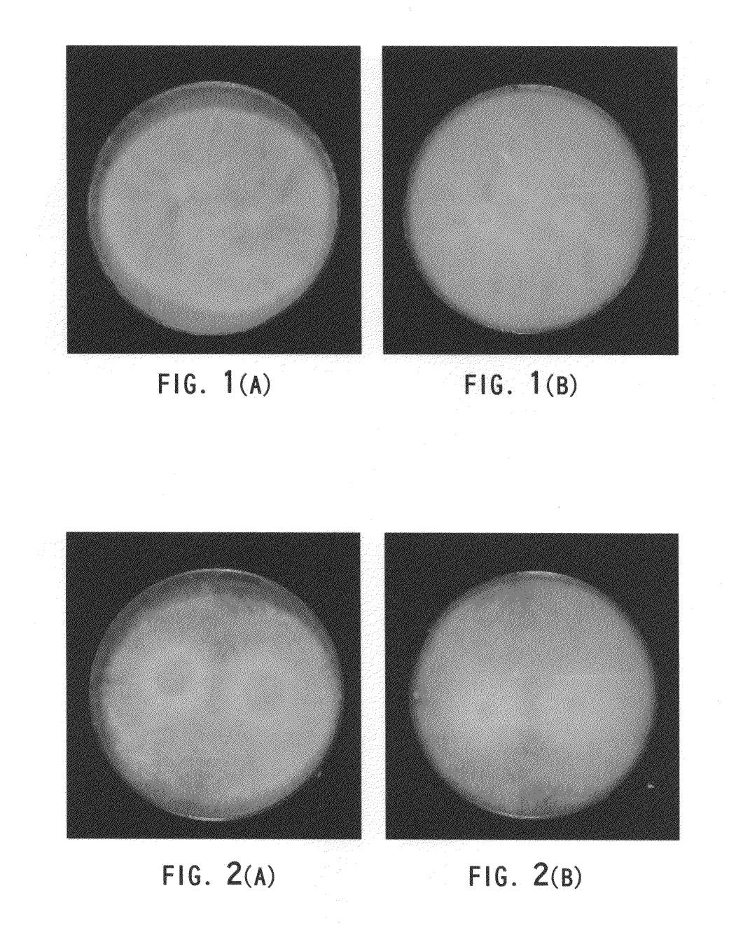

To briefly confirm a genetical new kind of variety, a method for investigating a reluctant reaction in a dual culture can be applied. That is, this method can clarify genetic difference of the new variety from the existing by observing the reluctant reaction in the dual culture. Consequently, PLE-3 and PLE-5 of the invention could be identified as new varieties by the dual culture method using the existing varieties (FIGS. 1(A) and 1(B)).

### [Verification Method]

To verify the reluctant reaction, the dual culture was prepared by placing fungal strains on potato dextrose agar medium at intervals of 3 cm in juxtaposition and cultivating the strains at 25 degrees C. for 28 days. The fungal strains used in the reluctant reaction experiment in the dual culture are shown in Table 1 below.

TABLE 1
Fungal strains used in the reluctant reaction experiment

| No. | Strain Name |
|---|---|
| — | HOKUTO PLE-2 |
| 07E2232 | PLE-3 |
| 07E2228 | PLE-5 |
| 1 | ATCC32047 |
| 2 | ATCC90212 |
| 3 | ATCC90787 |
| 4 | ATCC90887 |
| 5 | ATCC90888 |
| 6 | ATCC96054 |
| 7 | MH006062 (commercial strain: Fujita's Eryngii) |
| 8 | MH006200 (commercial strain: JA Fukuoka) |
| 9 | MH006202 (commercial strain: Yukiguni Maitake) |
| 10 | MH006207 (commercial strain: Masuda-Kashiwagi Mushroom) |
| 11 | MH006322 (commercial strain: Chokin-15) |
| 12 | MH006219 (commercial strain: Wild strain #1 grown in Italy) |
| 13 | MH006242 (commercial strain: Wild strain #2 grown in Italy) |
| 14 | MH006279 (commercial strain: Wild strain #1 grown in Spain) |

[Results:]

The results of the reluctant reaction experiment are shown in Table 2. In Table 2, the mark “+” denotes occurrence of reluctant reaction, and the mark “−” denotes no reluctant reaction.

All strains other than PLE-3 showed reluctant reaction relative to PLE-3 (07E2232) of the invention.

All strains other than PLE-5 showed reluctant reaction relative to PLE-5 (07E2228) of the invention.

TABLE 2
Results of dual culture

| Strain No. | PLE-3 | PLE-5 |
|---|---|---|
| 07E2232 (PLE-3) | − | + |
| HOKUTO PLE-2 | + | + |
| 07E2228 (PLE-5) | + | − |
| 1 (ATCC32047) | + | + |
| 2 (ATCC90212) | + | + |
| 3 (ATCC90787) | + | + |
| 4 (ATCC90887) | + | + |
| 5 (ATCC90888) | + | + |
| 6 (ATCC96054) | + | + |
| 7 (MH006062) | + | + |
| 8 (MH006200) | + | + |
| 9 (MH006202) | + | + |
| 10 (MH006207) | + | + |
| 11 (MH006322) | + | + |
| 12 (MH006219) | + | + |
| 13 (MH006242) | + | + |
| 14 (MH006279) | + | + |

+: Reluctant reaction occurred
−: No reluctant reaction occurred

(2) Cultural characteristics of PLE-3 and PLE-5:

The strains PLE-3 and PLE-5 of the invention were cultivated on the potato dextrose agar medium to be investigated in mycelial characteristics and so on. Consequently, the mycelia of all of the strains energetically grew. The growing rate of the mycelia at 20 degrees C. was about 3 mm a day (colony diameter). The strain having a flora surface of white (RHS: NN155C) could be obtained.

The strains were cultivated severally by 5 degrees C. in the range of 5 to 30 degrees C. When the strains were cultivated at 25 degrees C. for 14 days, PLE-5 grew somewhat poorly relative to PLE-3. The temperature for growing the strain PLE-3 best was 27 degrees C. differently from PLE-5 growing best at 28 degrees C.
(3) Morphologically characteristics of PLE-3 and PLE-5:

### [Cultivation Method]

(Examination Standard for Eryngii prescribed for plant variety registration based on Japanese Seeds and Seedlings Law was adopted.)

Bottle cultivation was performed using a polypropylene bottle having a bore diameter of 58 mm and a capacity of 850 ml. A culture medium was prepared by mixing cedar sawdust, bran and corn bran at the mixture ratio of 10:3:0.5 and regulating the water content to 65%. The cultivating bottle was filled up to the bottle shoulder with 510±10 grams of the culture media, and then, sterilized at high pressure. After the culture medium cooled, about 15 ml of sawdust seed fungus was implanted in the culture medium. The culture medium was controlled at 23 degrees C. and at a humidity of about 70%.

The culture medium was subjected to fungus scratching treatment after being cultivated for 40 days, and then, moved to an emergence room without dilution by water. The emergence room was controlled at a temperature of 17±1 degrees C., a humidity of 90%, an illumination intensity of 200 LUX and CO₂ concentration of approximately 2000 ppm. Mushrooms thus grown were harvested three days after the mushroom cap at the center of the carpophore spreads horizontally. The carpophore thus harvested was observed to determine the shape of its pileus. The results of verification based on Examination standard for Eryngii prescribed for plant variety registration based on Japanese Seeds and Seedlings Law will be described below.

### [Results]

Morphological comparison of PLE-3 and PLE-5:

The pileus of PLE-3 has a concave shape, but that of PLE-5 has a flat shape. There was a sharp difference in that PLE-3 has a thickness of 15.38 mm and PLE-5 has a thickness of 20.41 mm. PLE-3 and PLE-5 are similar in other pileus configuration.

With respect to the shape of hymenophorum, PLE-3 has a mushroom stem moderately attached to the pileus, and PLE-5 has a mushroom stem lightly attached to the pileus. PLE-3 and PLE-5 are similar in other hymenophorum feature.

The mushroom stem of PLE-3 has a relatively thick lower part, and that of PLE-5 is thick and long. The mushroom stem of PLE-3 is 79.41 mm in length, and that of PLE-5 is 86.22 mm in length. The mushroom stem of PLE-3 is 26.7 mm in diameter, and that of PLE-5 is 26.7 mm in diameter. Other features of PLE-3 and PLE-5 are approximately the same.

With respect to the other features than those noted above, PLE-3 has 3.14 carpophore stems on an effective average, and PLE-5 has 1.59 carpophore stems on an effective average. Further, a period from planting to peak of growing development and from fungus scratching to budding of PLE-5 was longer one day than that of PLE-3.

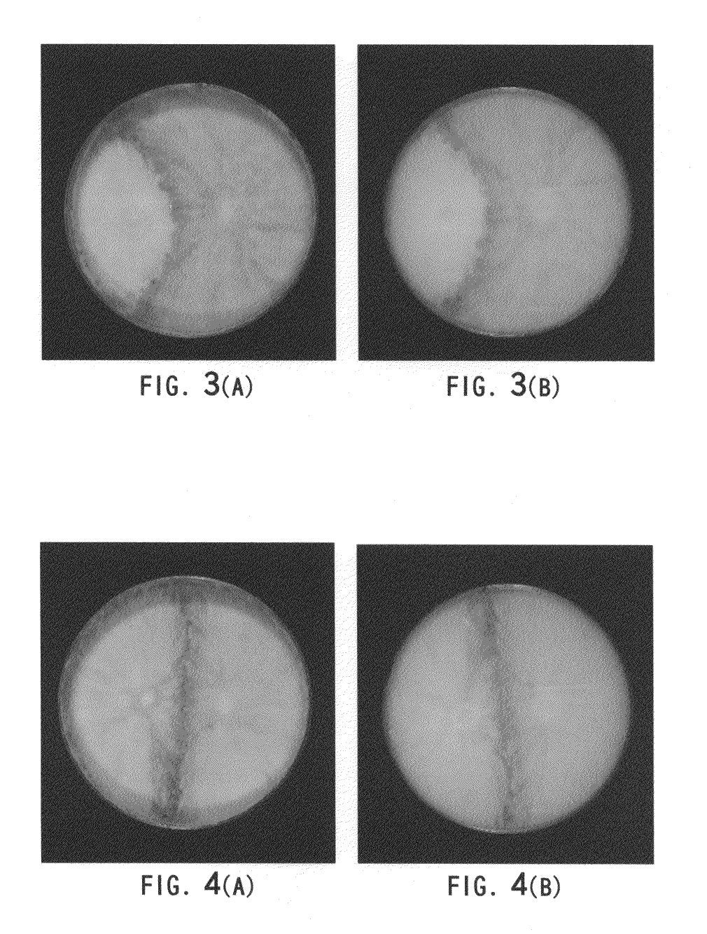

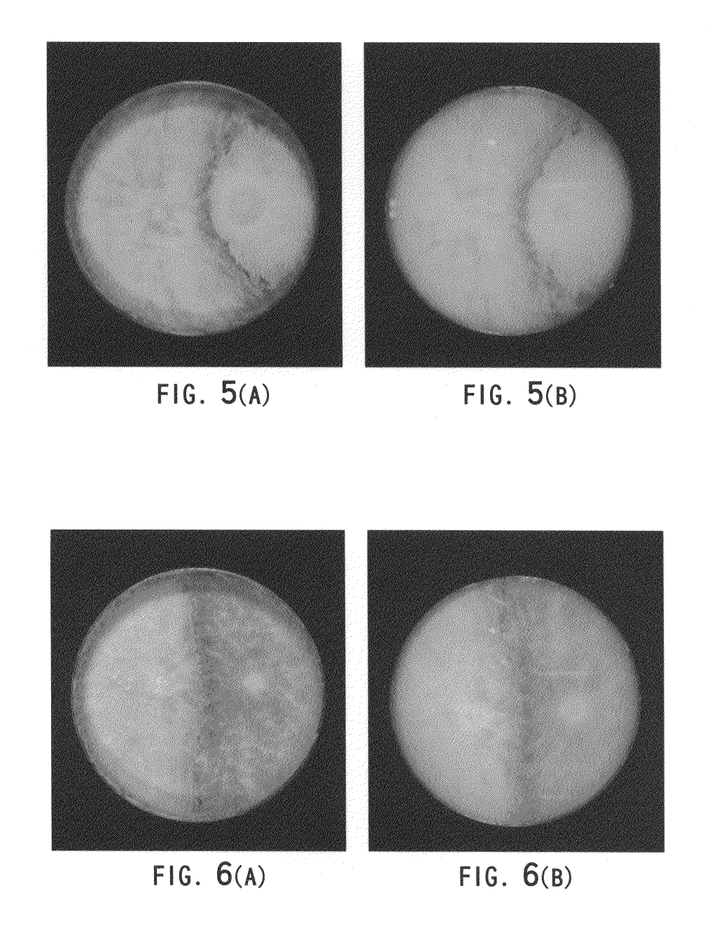

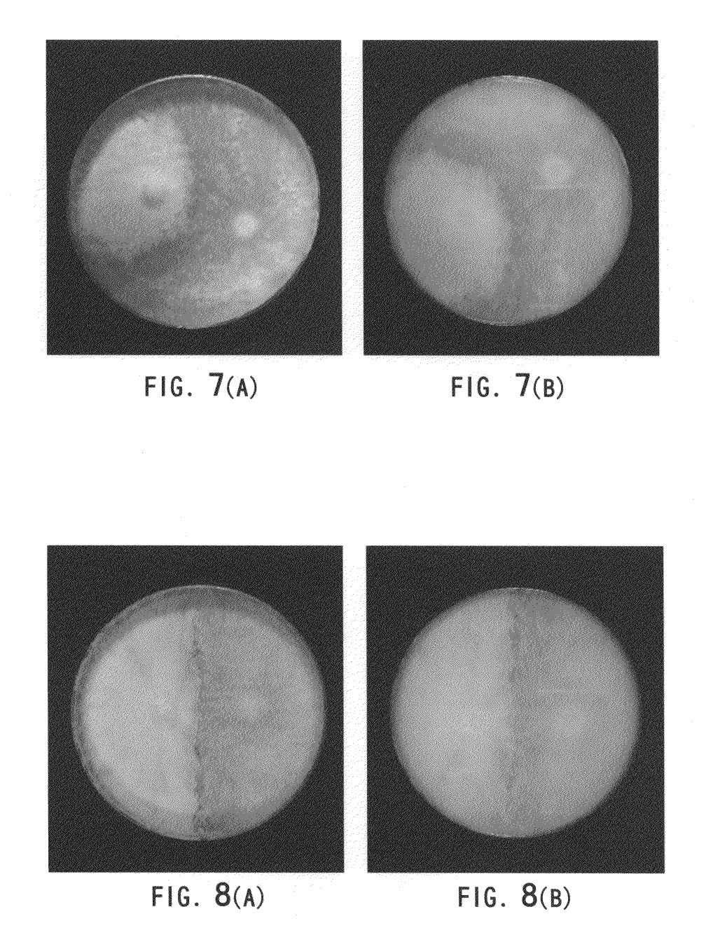

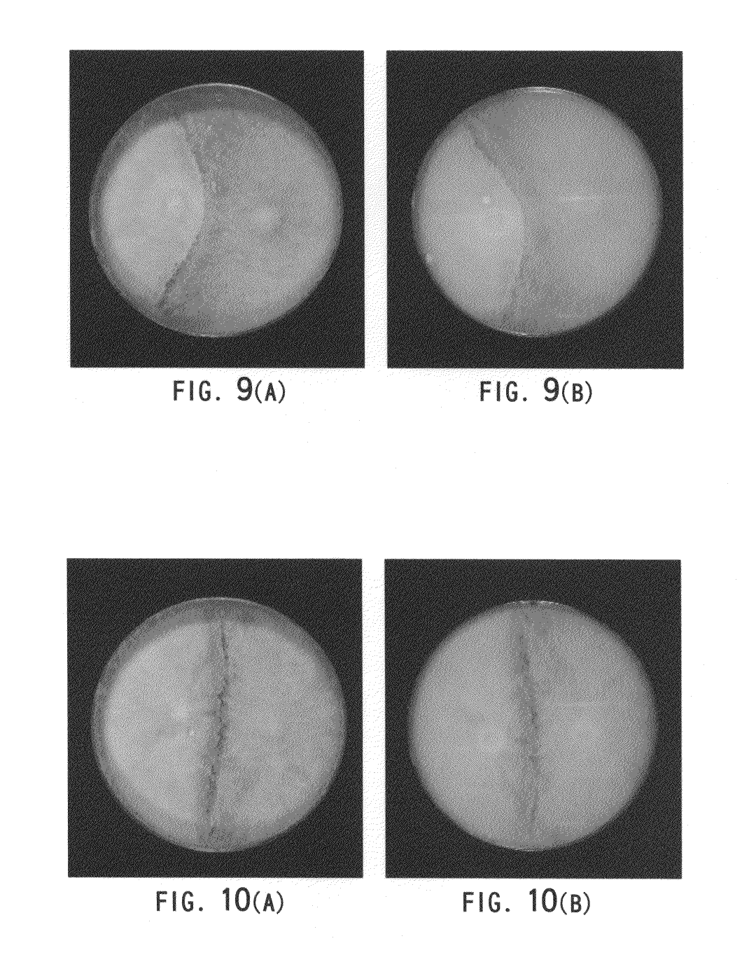

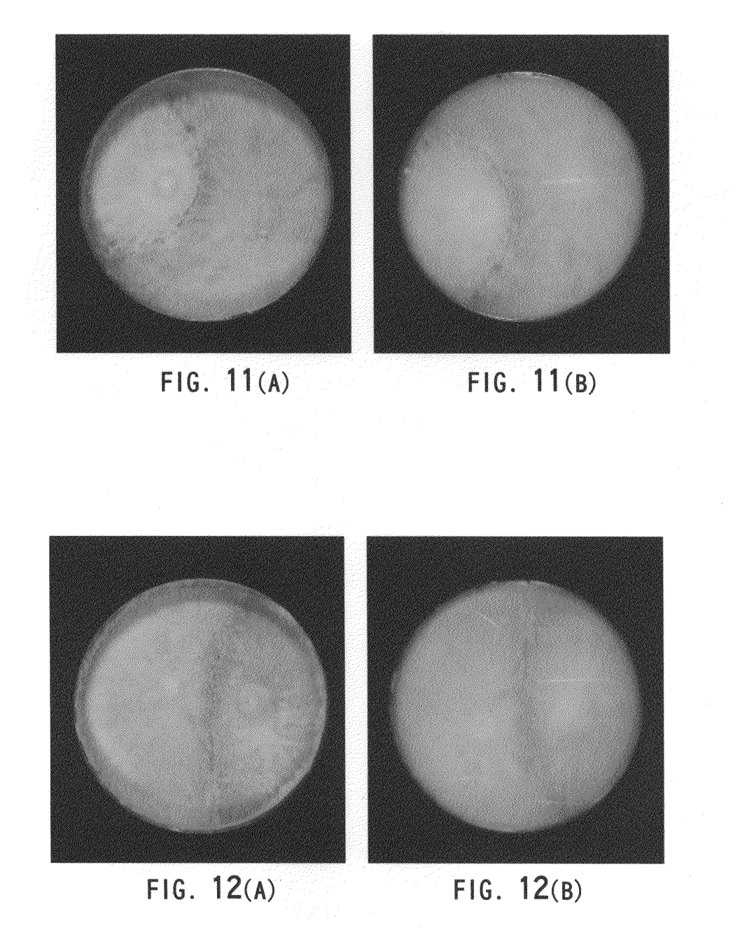

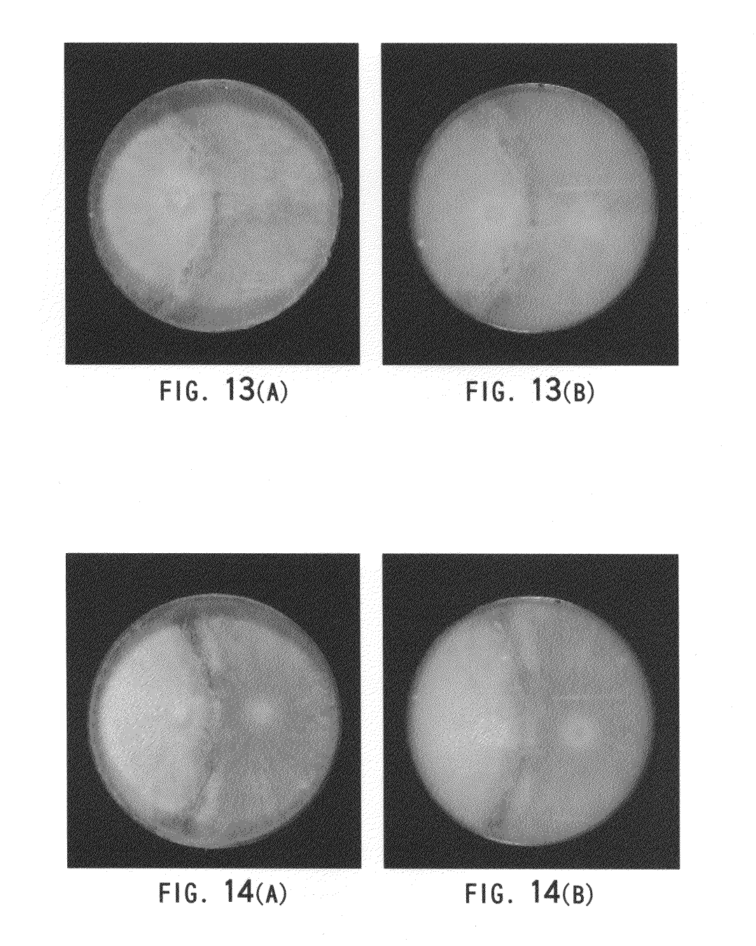

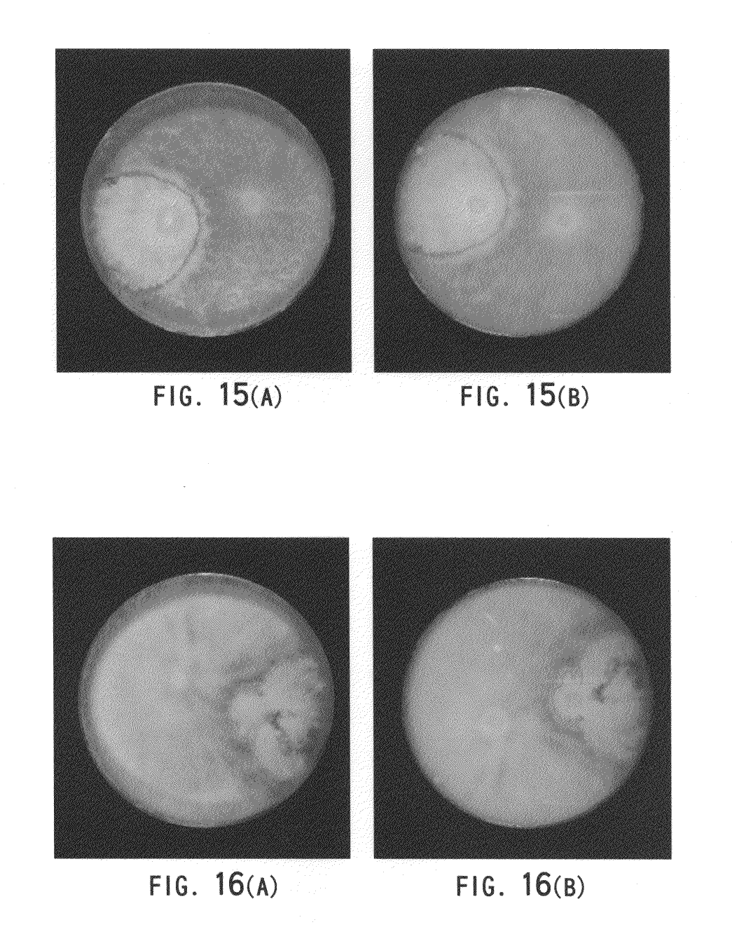

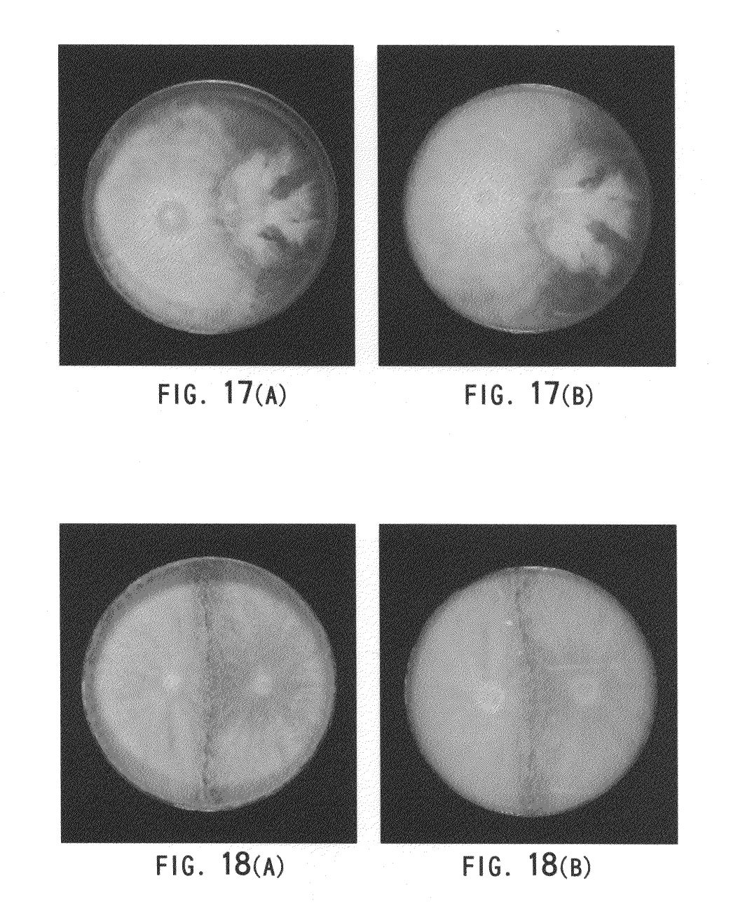

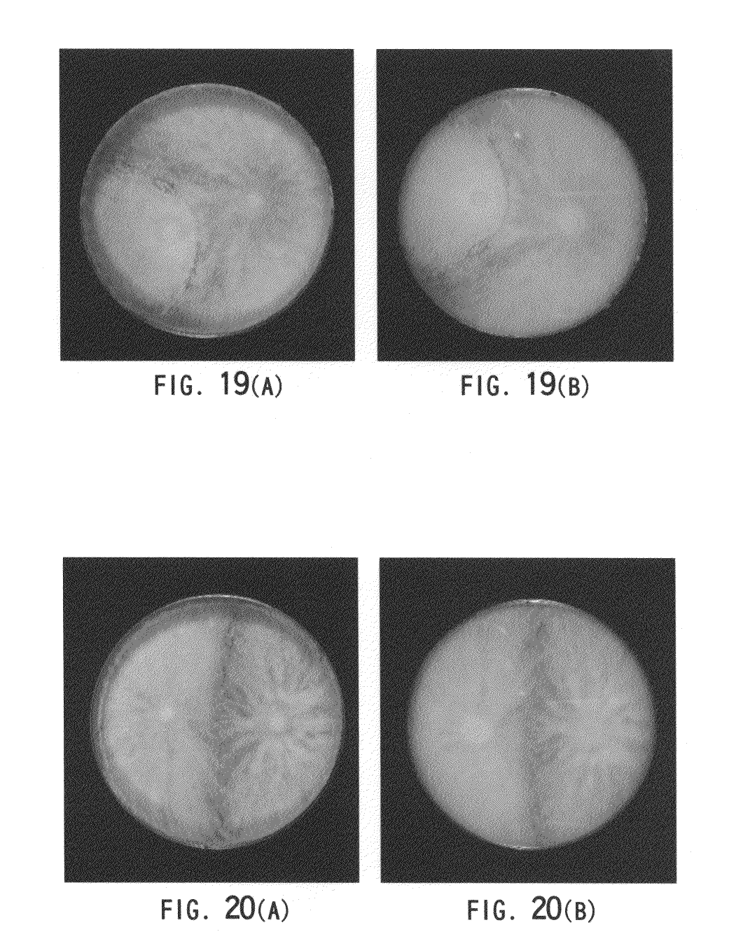

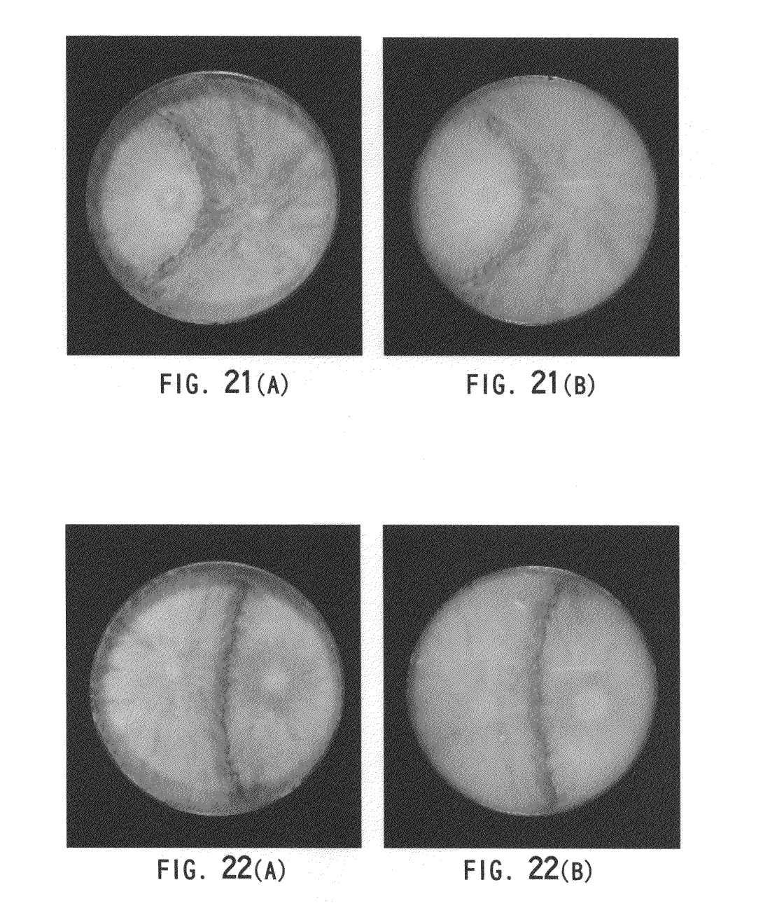

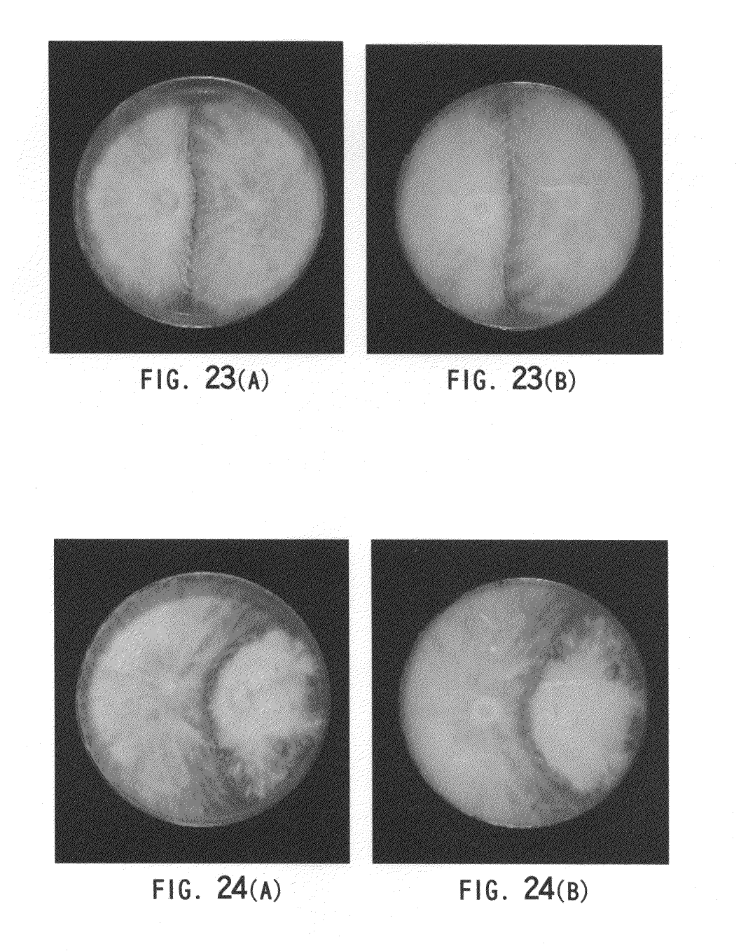

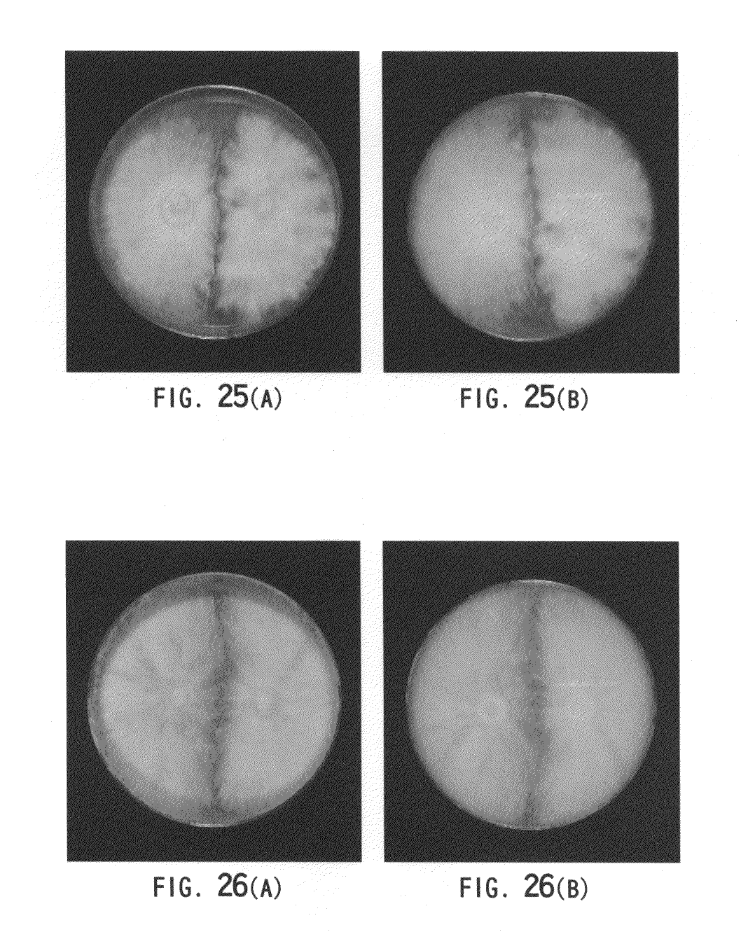

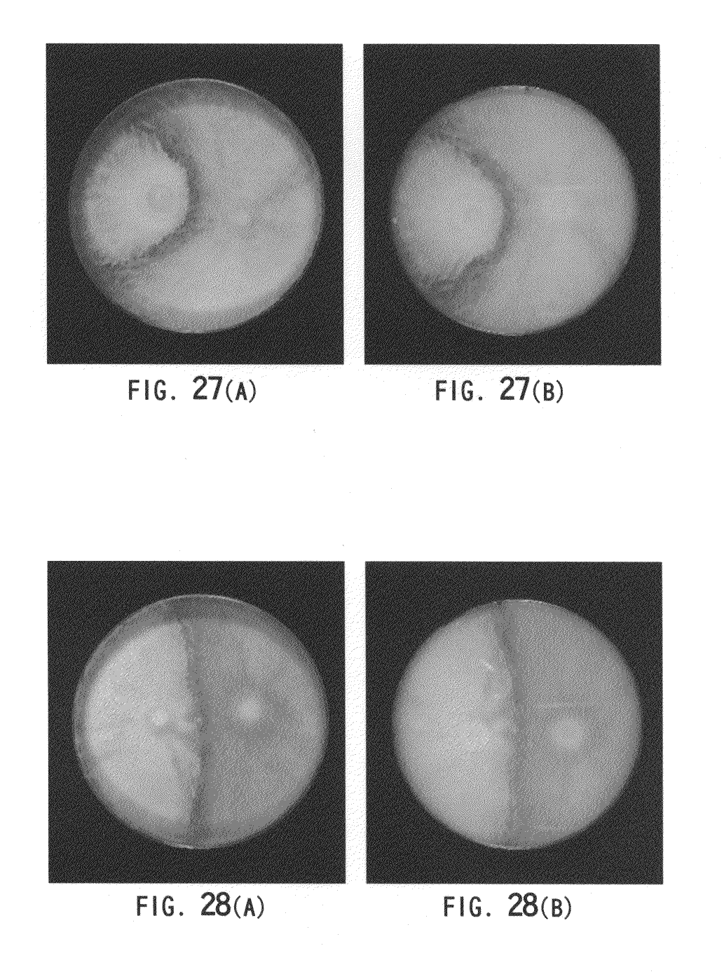

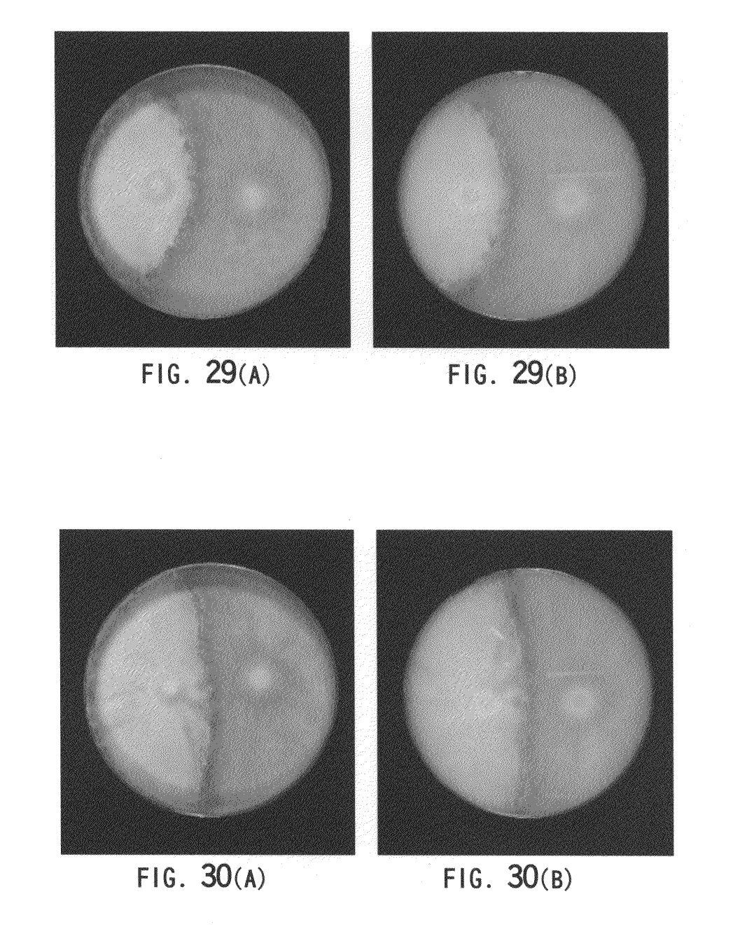

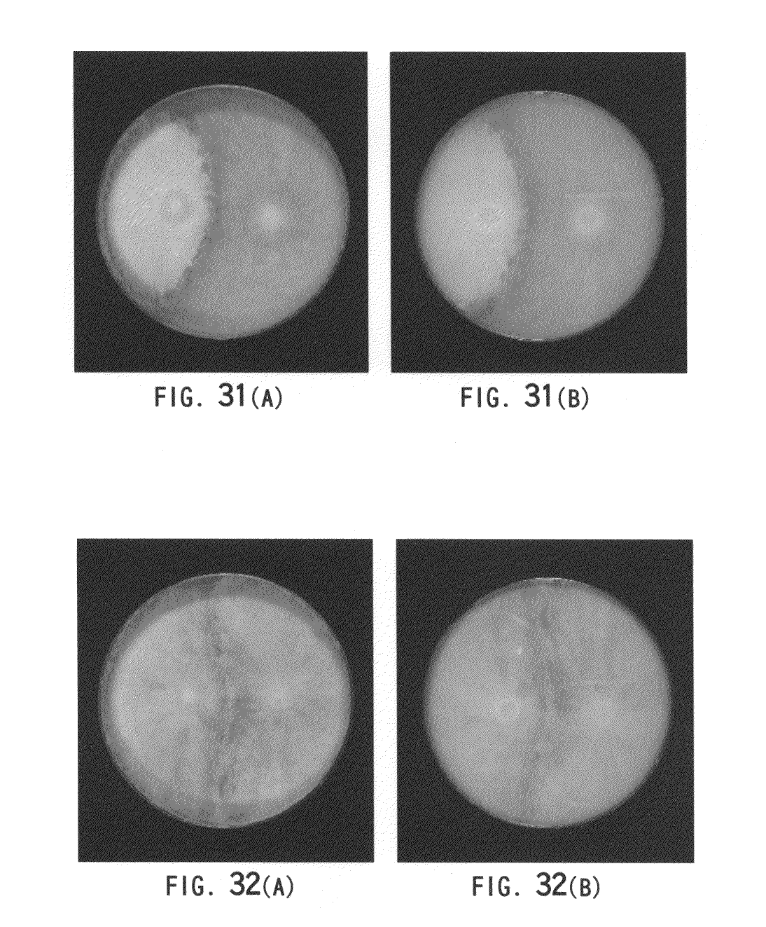

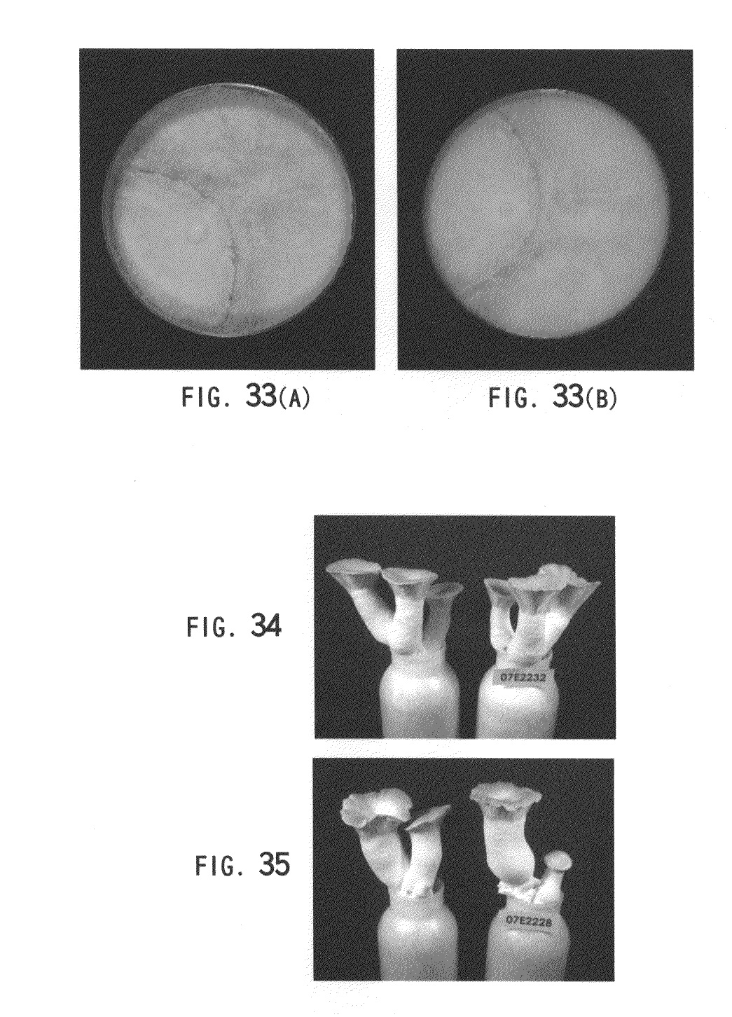

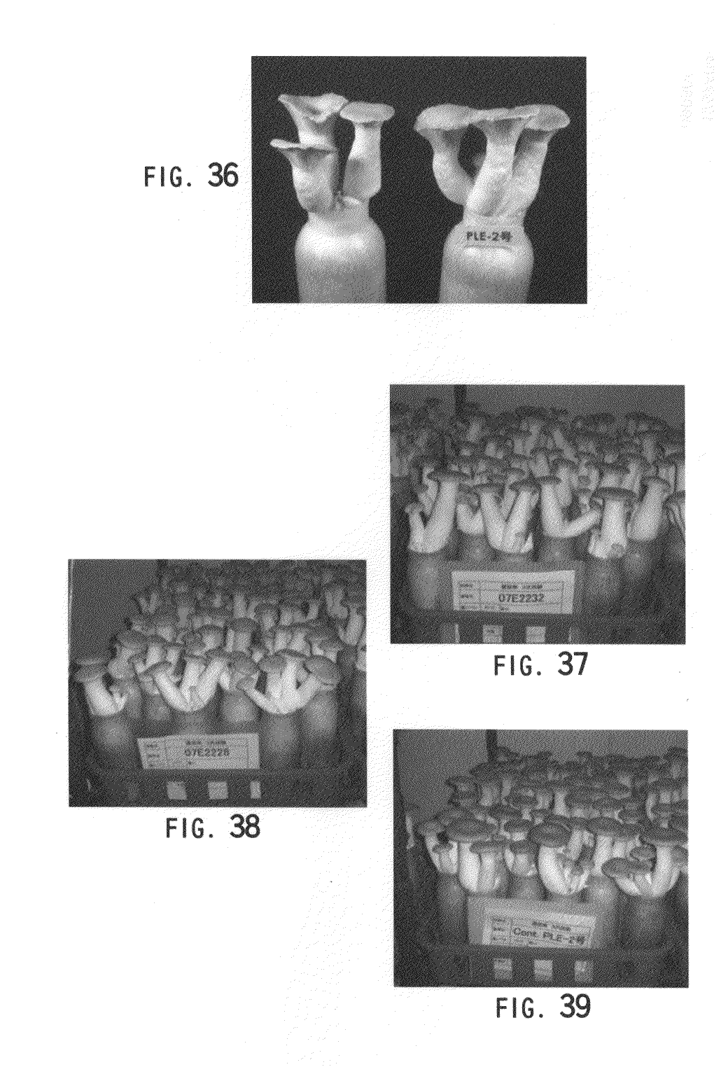

These characteristic features of the respective PLE-3 and PLE-5 are shown in FIG. 3 below.

TABLE 3
Characteristic Features of PLE-3 and PLE-5
(Characteristics of PLE-3 and PLE-5 and characteristics
of commercial similar variety HOKUTO PLE-2)

| | | | HOKUTO |
|---|---|---|---|
| Trait | PLE-3 | PLE-5 | PLE-2 |
| (1) Fungal Property | | | |
| Fungal density | Medium | Medium | Medium |
| Growth status of aerial | Normal | Normal | Normal |
| mycelia | | | |
| Peripheral shape of flora | Concentric | Concentric | Concentric |
| Color of upper surface of | White | White | White |
| flora | (RHS: | (RHS: | (RHS: |
| | N155C) | N155C) | N155C) |
| Color of lower surface of | Cream | Cream | Cream |
| flora | (RHS: 11D) | (RHS: 11D) | (RHS: 11D) |
| (2) Temperature Adaptability | | | |
| Optimum temperature of | 27 | 27 | 28 |
| mycelial growth (° C.) | | | |
| Growth rate at respective | | | |
| temperatures (mm/day) | | | |
| 5° C. | 0.45 | 0.38 | 0.35 |
| 10° C. | 1.18 | 0.97 | 1.46 |
| 15° C. | 1.8 | 1.72 | 1.96 |
| 20° C. | 2.95 | 2.65 | 3.73 |
| 25° C. | 6.04 | 4.87 | 6.05 |
| 30° C. | 5.22 | 4.63 | 5.96 |
| (3) Morphological Features | | | |
| Pileus | | | |
| Shape | Concave | Flat | Flat |
| Pattern | Streaky | Streaky | Streaky |
| Suffice protrusion | None | None | None |
| Size (mm) | 51.01 | 55.58 | 54.89 |
| Color of center part | (RHS: | (RHS: | (RHS: |
| | 199D) | 164D) | 199D) |
| Color of periphery | (RHS: | (RHS: | (RHS: |
| | 199D) | 164D) | 199D) |
| Thickness (mm) | 15.38 | 20.41 | 15.51 |
| Tenderness | Tough | Tough | Tough |
| Hymenophorum | | | |
| Alignment | Linear | Linear | Linear |
| Attaching intensity to stem | Moderate | Weak | Moderate |
| Width (mm) | 3.17 | 2.64 | 3.07 |
| Color | (RHS: | (RHS: | (RHS: |
| Stem | | | |
| Shape | Thick lower | Long slender | Thick lower |
| | part | | part |
| Attaching to pileus | Center part | Center part | Center part |
| Length (mm) | 79.41 | 86.22 | 86.87 |
| Ratio of pileus diameter to | 0.66 | 0.65 | 0.64 |
| stem length | | | |
| Diameter (mm) | 26.7 | 34.9 | 28.04 |
| Color | Pale yellow | Pale yellow | Pale yellow |
| | (RHS: | (RHS: | (RHS: |
| | 158D) | 158D) | 158D) |
| Tenderness | Tough | Tough | Tough |
| Growing development | | | |
| figure of carpophore | | | |
| Development figure | Monogenic | Monogenic | Monogenic |
| Effective stem number | 3.14 | 1.59 | 3.07 |
| Period required for | | | |
| carpophore development | | | |
| Period up to development | 64.15 | 65.09 | 64.71 |
| peak (day) | | | |
| Period from fungus | 24.15 | 25.09 | 24.71 |
| scratching to development | | | |
| (day) | | | |
| Temperature Adaptability | | | |
| Optimum temperature of | 17 | 17 | 17 |
| development (° C.) | | | |
| Optimum temperature of | 17 | 17 | 17 |
| growing (° C.) | | | |
| Yield (g) | 134.23 | 137.88 | 121.1 |

*“RHS” in the items related to color is short for Royal Horticultural Society, and the color codes found above are referred to the color charts stipulated by RHS.
Investigation place: Mushroom Laboratory, Hokuto Corporation of Shimokomazawa, Nagano-shi, Nagano, Japan
Investigation Period: 1 May 2008 to 20 Aug. 2008

(4) Embodiments in case of cultivation in a culture medium for commercial production:

The culture media prescribed as above were used herein to evaluate the varieties cultivated according to the invention and identify the morphological features of the varieties, but the culture media applicable for cultivating the object varieties have been improved to increase productivity of the subject mushrooms in an actual commercial production. Accordingly, the quality of the mushrooms actually produced may possibly not be consistent with the resultant data obtained from the cultivation experiment as described above. Thus, the mushrooms produced commercially in actuality according to the invention are demonstrated in FIGS. 37 through 39.

What is claimed is:

1. A new and distinct variety of mushroom plant as substantially illustrated or described in the specification.
